(19) United States
(12) Patent Application Publication
(10) Pub. No.: US 2022/0404350 A1
(43) Pub. Date: Dec. 22, 2022

(54) METHOD FOR DETERMINATION OF AGGREGATES

(71) Applicant: Cytiva Sweden AB, Uppsala (SE)

(72) Inventor: Tomas Dalmo, Uppsala (SE)

(21) Appl. No.: 17/776,200

(22) PCT Filed: Dec. 3, 2020
(86) PCT No.: PCT/EP2020/084366

§ 371 Date: May 11, 2022

(30) Foreign Application Priority Data
Dec. 3, 2019 (GB) 1917630.4

(51) Int. Cl. G01N 33/543; G01N 21/552

(52) U.S. Cl. CPC G01N 33/54373; G01N 21/553

(57) ABSTRACT

The present disclosure provides a method for determining of aggregates comprising one or more macromolecules, in a first sample potentially comprising aggregates of the macromolecule(s), comprising the steps of (a) contacting a first sample with a sensing surface of an interaction analysis sensor, said sensing surface having immobilised thereon a ligand comprising a hydrophobic group, which is capable of increased binding interaction with aggregates of macromolecule(s) compared to non-aggregated macromolecule(s); (b) determining at least one parameter for the interaction of the first sample with the sensing surface; (c) Performing at least one of steps (i) and (ii): (i) Comparing the at least one parameter determined in step (b) with the corresponding parameter(s) determined for at least one additional sample potentially comprising aggregates of the macromolecule(s); (ii) determining at least one parameter related to aggregate(s) of the macromolecule(s); and (d) determining the presence, fraction, concentration, and/or amount of macromolecule(s) in the form of aggregate(s) in the first sample. The present disclosure also relates to uses of said method and an interaction analysis sensor for use in said method, as well as an interaction analysis sensor and a method for determining of the stability of a macromolecule.

### FIELD OF THE DISCLOSURE

The present disclosure is directed to a method for determining of aggregates comprising one or more macromolecules in a sample, uses of said method and an interaction analysis sensor for use in said method. The present disclosure also relates to an interaction analysis sensor and a method for determining of the stability of a macromolecule.

### BACKGROUND OF THE DISCLOSURE

Bio-macromolecules, such as proteins, nucleic acids and polysaccharides, may often partially occur in the form of aggregates, or multimers, such as dimers, trimers or higher oligomers. Within the field of biological production of recombinant proteins, where desired polypeptides or proteins are produced in host organisms and isolated from cells or cell extracts under conditions and in concentrations quite different from those in their natural environment, the conditions may favor the formation of such aggregates through intermolecular disulphide linkages or other covalent bonds, or through non-covalent interactions. The presence of such aggregates of a target macromolecule is many times undesired. Protein aggregation is thus a common issue encountered during bioprocess development and manufacturing of biotherapeutics. Aggregated forms of a macromolecule may have lower biological activity than the non-aggregated form of the macromolecule; it may even completely lack the desired biological activity or may cause undesired side-effects. Hence, it is essential for therapeutic safety that a therapeutic protein is in a non-aggregated state and that there are no aggregates of molecules present. It is consequently of importance that the amount of aggregates produced during cell culturing and/or during the subsequent purification process can be controlled.

Formulation development and protein stability are among the cornerstones of a drug development process, in which it is required to obtain long shelf-life and maintain potency of drug candidates. To evaluate the stability of a protein, its melting process and/or aggregation level is measured. Among the currently used technologies for measurement of melting process and/or aggregation level are fluorimetry, calorimetry and light scattering. Fluorimetry is widely used due to its simplicity and is based on the affinity of certain fluorescent probes towards protein aggregates. Such probes are e.g. Sypro orange and Thioflavin T. Computer simulations on Thioflavin T have shown that it is mainly the interaction with hydrophobic side-chains of amino acids that enables the detection with the dye (M. Biancalana, S. Koide, Molecular mechanism of Thioflavin-T binding to amyloid fibrils, Biochimica et Biophysica Acta 1804(7): 1405-1412, 2010).

Analytical sensor systems that can monitor interactions between molecules, such as biomolecules, in real time are often based on optical biosensors and usually referred to as interaction analysis sensors or biospecific interaction analysis sensors. A representative such biosensor system is the BIACORE® instrumentation (GE Healthcare Bio-Sciences AB, Uppsala, Sweden), which uses surface plasmon resonance (SPR) for detecting interactions between molecules in a sample and molecular structures immobilised on a sensing surface.

A detailed discussion of the technical aspects of the BIACORE® instruments and the phenomenon of SPR may be found in U.S. Pat. No. 5,313,264. More detailed information on matrix coatings for biosensor sensing surfaces is given in, for example, U.S. Pat. Nos. 5,242,828 and 5,436,161. In addition, a detailed discussion of the technical aspects of the biosensor chips used in connection with the BIACORE® instruments may be found in U.S. Pat. No. 5,492,840. All of the above-mentioned patents are incorporated by reference in their entirety herein.

In WO2011/093782 A1, incorporated by reference in its entirety herein, the BIACORE® system has been described in the context of a method for determination of aggregates of macromolecule monomers by use of a ligand having specific binding to the analyte of interest, i.e. the macromolecule. More particularly, protein A is used as a ligand, for specific binding to the Fc portion of antibodies. The method may be used for purification of macromolecules.

However, there is a need in the art for alternative methods, as well as improved for the determination of aggregates of macromolecule in a sample.

### SUMMARY OF THE DISCLOSURE

The above objective to provide alternative and improved methods for the determination of aggregates of macromolecule in a sample is achieved by the present disclosure, which is directed to a method based on the inventor's realization of certain properties of degraded macromolecules, which led to the conclusion that novel types of ligands may be used for detection of aggregates of macromolecules.

More particularly, the presently disclosed method for determining of aggregates comprising one or more macromolecules, in a first sample potentially comprising aggregates of the macromolecule(s), comprises the steps of:
a) contacting a first sample with a sensing surface of an interaction analysis sensor, said sensing surface having immobilised thereon a ligand comprising a hydrophobic group, which is capable of increased binding interaction with aggregates of macromolecule(s) compared to non-aggregated macromolecule(s);
b) determining at least one parameter for the interaction of the first sample with the sensing surface;
c) Performing at least one of steps (i) and (ii):
   i) Comparing the at least one parameter determined in step (b) with the corresponding parameter(s) determined for at least one additional sample potentially comprising aggregates of the macromolecule(s);
   ii) determining at least one parameter related to aggregate(s) of the macromolecule(s); and
d) determining the presence, fraction, concentration, and/or amount of macromolecule(s) in the form of aggregate(s) in the first sample.

The present disclosure further provides a method for determining of the stability of a macromolecule, comprising performing the above-described method, including step (c)(i), wherein the additional sample is a second sample, for which the presence, fraction, concentration, and/or amount of macromolecule(s) in the form of aggregate(s) is to be determined.

Further provided are various uses of the herein disclosed method, more particularly:
A use of the method for the determination of the stability of a macromolecule;
A use of the method for the determination of degradation of protein in a sample;
A use of the method for the determination of biological activity of a protein drug or a polypeptide drug;
A use of the method for the quantitative determination of the fraction, concentration, and/or amount of aggregated macromolecule(s) in a sample;
A use of the method for the qualitative determination of the presence, fraction, concentration, and/or amount of aggregated macromolecule(s) in a sample.

The present disclosure also provides an interaction analysis sensor for use in the herein disclosed method.

Also provided is an interaction analysis sensor comprising a sensing surface, on which sensing surface is immobilised a ligand comprising a hydrophobic group, which is capable of increased binding interaction with aggregates of macromolecule(s) compared to non-aggregated macromolecule(s).

Preferred aspects of the present disclosure are described below in the detailed description and in the dependent claims.

### BRIEF DESCRIPTION OF THE DRAWINGS

FIG. 1 is a flow chart of a method for determining of aggregates of macromolecules in a sample according to the present disclosure.

FIG. 2 schematically shows a non-limiting example of an interaction analysis sensor, which may be used to perform the method according to the present disclosure.

FIG. 3 depicts a sensorgram illustrating results of Example 1, obtained by use of a BIACORE® system.

FIG. 4 shows that the results obtained in Example 1 by use of the BIACORE® system correlate with the results obtained by use of the prior art method of fluorimetry.

FIG. 5 depicts a sensorgram illustrating results of Example 2, obtained by use of a BIACORE® system

FIG. 6 depicts a sensorgram illustrating results of Example 5, obtained by use of a BIACORE® system

FIG. 7 is a schematic view of the methodology described in Example 6.

### DETAILED DESCRIPTION OF THE DISCLOSURE

As mentioned above, the present disclosure relates to the detection and analysis of multimeric forms, or aggregates, of a macromolecule, typically a protein, such as an antibody, in a sample, typically a fluid sample. In brief, the method is based on utilizing differences in the affinity of a ligand, immobilised on a sensing surface of a biomolecular interaction analysis sensor, for non-aggregated macromolecule and aggregates of the macromolecule, respectively. Because of said differences in affinity, the kinetics of the binding interaction of the non-aggregated macromolecule with the ligand will be different from the kinetics of the binding interaction of aggregates of the macromolecule with the ligand. As described in more detail elsewhere herein, the presence (or fraction, concentration or amount) of aggregate of a macromolecule in the sample may be determined following determination of parameters (e.g. kinetic parameters) of the binding interaction between the macromolecule (aggregated and/or non-aggregated) and the immobilised ligand.

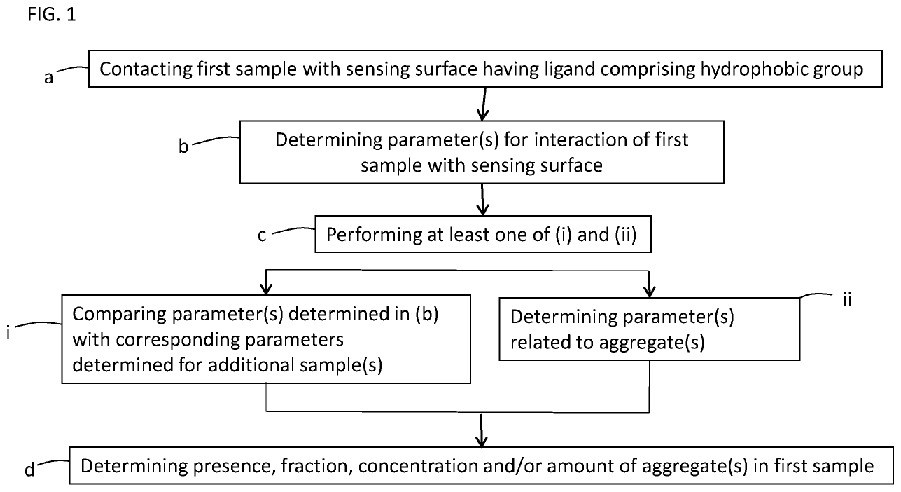

The present disclosure solves or at least mitigates the problems associated with existing methods for determining of aggregates of macromolecules in a sample by providing, as illustrated in FIG. 1, a method for determining of aggregates comprising one or more macromolecules, in a first sample containing the macromolecule(s), comprising the steps of:
a) contacting a first sample with a sensing surface of an interaction analysis sensor, said sensing surface having immobilised thereon a ligand comprising a hydrophobic group, which is capable of increased binding interaction with aggregates of macromolecule(s) compared to non-aggregated macromolecule(s);
b) determining at least one parameter for the interaction of the first sample with the sensing surface;
c) Performing at least one of steps (i) and (ii):
   i) Comparing the at least one parameter determined in step (b) with the corresponding parameter(s) determined for at least one additional sample potentially comprising aggregates of the macromolecule(s);
   ii) determining at least one parameter related to aggregate(s) of the macromolecule(s); and
d) determining the presence, fraction, concentration, and/or amount of macromolecule(s) in the form of aggregate(s) in the first sample.

The term “macromolecule” has its conventional meaning in the field of bioprocessing, in which macromolecules are produced (often recombinantly) by cells in a cell culture and purified from the cell culture by any means of separation and purification. Alternatively, the macromolecules are present in a biological solution which does not necessarily originate from a cell culture. Non-limiting examples of macromolecules are biomacromolecules, which are large biological polymers that are made up of monomers linked together, e.g. peptides and proteins (which can be native or recombinant), including but not limited to enzymes, antibodies and antibody fragments, as well as carbohydrates, and nucleic acid sequences, such as DNA and RNA. The macromolecule for which the presence of aggregates in a preparation of the macromolecule may be determined by the method of the present disclosure is typically a protein or polypeptide, particularly a therapeutic protein or polypeptide, such as an antibody, but may also be, for example, a nucleic acid. A macromolecule or a biomacromolecule may for example be a biopharmaceutical, i.e. a biological molecule, including but not limited to a biological macromolecule, which is intended for use as a pharmaceutical compound.

It is to be understood that “a macromolecule” is intended to mean a type of macromolecule and that the singular form of the term may encompass a large number of individual macromolecules, or specimens, of the same type.

Herein, the term “non-aggregated macromolecule” is intended to mean a non-degraded macromolecule. A non-aggregated macromolecule may herein alternatively be called “non-degraded macromolecule” or “intact macromolecule”. In a typical embodiment herein, in which the macromolecule is a protein or a polypeptide, the non-aggregated macromolecule may be described as having an essentially intact tertiary structure, which usually involves an essentially hydrophilic surface of the macromolecule, while hydrophobic moieties are located in the interior of the macromolecule. Hence, a non-aggregated macromolecule essentially does not have hydrophobic moieties or hydrophobic groups exposed on the surface.

In contrast, in a protein or polypeptide which starts to degrade, the tertiary structure is gradually destroyed, which exposes hydrophobic moieties to the environment surrounding the protein or polypeptide. A protein or polypeptide macromolecule which is being degraded, or has been degraded, may form aggregates. A non-aggregated form of a macromolecule is in a monomeric state. Aggregates of a macromolecule may contain multimeric forms of the macromolecule, such as dimers, trimers etc. of the macromolecule. An individual macromolecule which is degrading may form aggregates with other individual, degrading, specimens of the same type of macromolecule, and/or may form aggregates with individual, degrading, specimens of other types of degrading macromolecules, or a combination thereof. Since aggregates of macromolecules contain degrading macromolecules, it follows that aggregates of macromolecules have hydrophobic moieties exposed on their surfaces.

Herein, the term “hydrophobic moiety” is intended to mean a hydrophobic part of the macromolecule or a hydrophobic group present in the macromolecule.

In this context, “increased binding interaction” means that a ligand comprising a hydrophobic group is capable of at least 50%, such as 60%, 70%, 80%, 90%, 95%, 96%, 97%, 98%, 99%, or 100%, more binding to aggregates of macromolecule than to non-aggregated macromolecule. In other words, a ligand comprising a hydrophobic group has at least 50%, such as 60%, 70%, 80%, 90%, 95%, 96%, 97%, 98%, 99%, or 100%, higher affinity for aggregates of macromolecule than for non-aggregated macromolecule, due to the properties of the ligand's hydrophobic group.

It is to be understood that the term “sample” as used herein encompasses any type of sample obtainable from a cell culture, or from a fluid originating from a cell culture which fluid is at least partly purified, by any means of separation and purification, or any type of sample obtainable from a biological solution.

Herein, the term “cell culture” refers to a culture of cells or a group of cells being cultivated, wherein the cells may be any type of cells, such as bacterial cells, viral cells, fungal cells, insect cells, or mammalian cells. A cell culture may be unclarified, i.e. comprising cells, or may be cell-depleted, i.e. a culture comprising no or few cells but comprising biomolecules released from the cells before removing the cells. Further, an unclarified cell culture as used in the presently disclosed method may comprise intact cells, disrupted cells, a cell homogenate, and/or a cell lysate.

The term “biological solution” is intended to mean a solution of biological origin, comprising a biomolecule or a mixture of several types of biomolecules. Examples of biological solutions are any type of bodily fluid originating from a human or an animal, such as plasma, blood, sputum, urine, and milk.

The term “antibody” as used herein means an immunoglobulin (IgG) which may be natural or partly or wholly synthetically produced. The term includes, but is not limited to, active fragments, including Fab antigen-binding fragments, univalent fragments and bivalent fragments. The term also covers any protein having a binding domain which is homologous to an immunoglobulin binding domain. Such proteins can be derived from natural sources, or partly or wholly synthetically produced.

Exemplary antibodies are the immunoglobulin isotypes and the Fab, Fab′, F (ab′) 2, scFv, Fv, dAb, and Fd fragments. The method of the present invention for determination of the presence, fraction, concentration or amount of aggregate in, for example, a therapeutic antibody preparation may be used to monitor aggregate formation during process development in order to optimize procedures for attaining a high-quality end product. The presence of aggregates in therapeutic antibody preparations generally have a negative impact on patient safety and must be effectively controlled during process manufacturing.

The interaction analysis sensor used in the presently disclosed methods is typically a biosensor. As is well known, a biosensor is typically based on label-free techniques, detecting a change in a property of a sensor surface, such as mass, refractive index or thickness of the immobilised layer. Typical biosensors for the purposes of the present invention are based on mass detection at the sensor surface and include especially optical methods and piezoelectric or acoustic wave methods. Representative sensors based on optical detection methods include those that detect mass surface concentration, such as sensors based on reflection-optical methods, including e.g. evanescent wave-based sensors, such as surface plasmon resonance (SPR) sensors; frustrated total reflection (FTR) sensors, and waveguide sensors, including e.g. reflective interference spectroscopy (RIfS) sensors. Piezoelectric and acoustic wave sensors include surface acoustic wave (SAW) and quartz crystal microbalance (QCM) sensors.

Biosensor systems based on SPR and other detection techniques are commercially available today. Exemplary such SPR biosensors include the above-mentioned flow-through cell-based BIACORE® systems and ProteOn™ XPR system (Bio-Rad Laboratories, Hercules, Calif., USA) which use surface plasmon resonance for detecting interactions between molecules in a sample and molecular structures immobilised on a sensing surface.

Herein, such molecular structures immobilised on the sensing surface will be denoted “ligand”. The term “ligand” may be used interchangeably with the terms “specific binding molecule”, “specific binding partner”, “capturing molecule” and “capturing agent”. In the following, the molecules in the sample which interact with a ligand on the sensing surface are referred to as “analyte”. As sample is passed over the sensing surface, analytes in the sample bind to the ligands on the sensing surface, which leads to a change in concentration and consequently a change in refractive index at the sensing surface. the progress of binding between the analyte and the ligand is directly reflected by changes in refractive index at the sensing surface that correspond to changes in signal intensity during the binding. Injection of the sample is followed by a buffer flow during which the detector response reflects the rate of dissociation of the complex of analyte and ligand on the surface. A typical output from the BIACORE® system is a graph or curve describing change in refractive index at the sensing surface and thereby the progress of the molecular interaction with time, including an association phase part and a dissociation phase part. This graph or curve, which is usually displayed on a computer screen, is often referred to as a binding curve or “sensorgram”, in which the vertical axis (y-axis) indicates the response and the horizontal axis (x-axis) indicates the time. In the BIACORE® system, the SPR response values are expressed in resonance units (RU). One RU represents a change of 0.0001° in the angle of minimum reflected light intensity, which for most proteins and other biomolecules correspond to a change in concentration of about 1 pg/mm^ on the sensing surface.

In this context, “ligand” is a molecule that has a known or unknown affinity for a given analyte and includes any capturing agent immobilised on the surface, whereas “analyte” includes any specific binding partner to the ligand.

The analytes of interest according to the present disclosure are macromolecules, more particularly aggregates of macromolecules. Consequently, herein the terms “analyte” and “aggregate of macromolecule” may be used interchangeably.

The phenomenon of SPR is well known, suffice it to say that SPR arises when light is reflected under certain conditions at the interface between two media of different refractive indices, and the interface is coated by a metal film, typically silver or gold. In the BIACORE® instruments, the media are the sample and the glass of a sensor chip, which is contacted with the sample by a micro fluidic flow system. The metal film is a thin layer of gold on the chip surface. SPR causes a reduction in the intensity of the reflected light at a specific angle of reflection. This angle of minimum reflected light intensity varies with the refractive index close to the surface on the side opposite from the reflected light; in the BIACORE® system this is the sample side.

With the BIACORE® system (and analogous sensor systems) it is possible to determine, in real time, without the use of labeling, and often without purification of the substances involved, not only the presence and concentration of a particular molecule, or analyte, in a sample, but also additional interaction parameters, such as various kinetic parameters, including kinetic rate constants for association (binding) and dissociation in the molecular interaction as well as the affinity for the interaction on the sensing surface between analyte and ligand. The association rate constant (kₐ) and the dissociation rate constant (IQ) can be obtained by fitting the resulting kinetic data for a number of different sample analyte concentrations to mathematical descriptions of interaction models in the form of differential equations. The affinity (expressed as the affinity constant K_{A} or the dissociation constant K_{D}) can be calculated from the association and dissociation rate constants.

In the method according to the present disclosure, kinetic parameters of particular interest to determine may be selected from the group consisting of the association rate (alternatively called “binding rate” or “on-rate”), the dissociation rate (alternatively called “off-rate”), the association rate constant (kₐ), the dissociation rate constant (IQ), the affinity constant (K_{A}), and the dissociation constant (K_{D}). Another parameter of interest to determine is the binding level of analytes, or more specifically the amount, fraction or concentration of analytes bound to the ligands on the sensing surface, e.g. just before dissociation starts as well as at the end of dissociation.

With regard to the association phase, the on-rate may be determined as the initial binding rate, represented by the initial slope of the binding curve. The slope is typically determined during a short time window, shortly (typically a few seconds) after association has started, and usually expressed as resonance units or response units per second (RU/s). The aggregates of macromolecule and non-aggregated macromolecule, respectively, exhibit very different on-rates to the ligand on the sensor surface, due to the above-mentioned increased binding interaction of the ligand (due to the properties of the ligand's hydrophobic group) with aggregates of macromolecule compared to non-aggregated macromolecule. As defined above, in this context, “increased binding interaction” means that the ligand (due to the properties of the ligand's hydrophobic group) has at least 50%, such as 60%, 70%, 80%, 90%, 95%, 96%, 97%, 98%, 99%, or 100%, higher affinity for aggregates of macromolecule than for non-aggregated macromolecule. Further, the ligand ideally has a very low binding interaction with non-aggregated macromolecule, which results in a very low initial slope on the binding curve, i.e. a very low on-rate. In other words, the ligand ideally has a very low affinity for non-aggregated macromolecule, which is herein defined as meaning that the ligand's affinity for non-aggregated macromolecule is a maximum of 50%, such as 40%, 30%, 20%, 10%, 5%, 4%, 3%, 2%, 1%, or 0% of the ligand's affinity for aggregates of macromolecule (due to the properties of the ligand's hydrophobic group). In the experiments described elsewhere herein, the ligand's affinity for non-aggregated macromolecule was only about 0-1.2% of its affinity for aggregates of the macromolecule.

In case of a reaction-controlled interaction at the sensing surface (i.e. in the absence of any mass transfer limitation), an aggregate will give a greater response, i.e. faster on-rate, at a mass sensing surface than a non-aggregated macromolecule. The greater the fraction of aggregate is in a sample comprising both non-aggregated and aggregated macromolecule, the greater the initial slope will be, and therefore the more the initial slope will differ from that determined for a sample containing only non-aggregated macromolecule. If the on-rate has been determined in this way for a number of samples with different fractions of aggregate, the aggregate fraction in an unknown sample may thus be determined.

In the dissociation phase (i.e. when the surface is no longer exposed to sample and dissociation from the surface may take place), the above-mentioned stronger binding of aggregate to the surface compared to a non-aggregated macromolecule due to the ligand's higher affinity for the aggregate causes a slower “off-rate” for the aggregates. Therefore, the greater the fraction of aggregate is in a sample comprising both non-aggregated and aggregated macromolecule, the slower is the off-rate, and therefore the more the off-rate will differ from that determined for a sample containing only non-aggregated macromolecule. The off-rate may, for example, be represented by the residual binding level (response) at a predetermined time after dissociation has been initiated. Provided that the off-rate has been determined for a number of samples with different fractions of aggregate, the aggregate fraction in an unknown sample may thus be determined.

For a qualitative determination of the presence of aggregate(s) in a sample, it is sufficient to compare the determined parameter (e.g. kinetic parameter) with that of a sample comprising 100% non-aggregated macromolecule.

For the type of measurements concerned herein it is understood that, generally, a low saturation level at the sensing surface will enable a high sample throughput, whereas higher levels will facilitate detection of low fractions of aggregates in the sample.

The term “hydrophobic group” as used herein is defined as a group of molecules which has a log P value>0. The partition coefficient, abbreviated P, is defined as a particular ratio of the concentrations of a solute between the two solvents (a biphase of liquid phases), specifically for un-ionized solutes, and the logarithm of the ratio is thus log P. When one of the solvents is water and the other is a non-polar solvent, then the log P value is a measure of lipophilicity or hydrophobicity. The defined precedent is for the lipophilic and hydrophilic phase types to always be in the numerator and denominator respectively; for example, in a biphasic system of n-octanol (hereafter simply “octanol”) and water:

$$log {P}_{oct / wat} = log ( \frac{{[ solute ]}_{octanol}^{un - ionized}}{{[ solute ]}_{water}^{un - ionized}} ) .$$

A log P value<0 indicates that a higher percentage of the solute is in the hydrophilic phase. Conversely, a log P value>0 indicates a higher percentage of the solute in the lipophilic phase, i.e. the hydrophobic phase.

According to the present disclosure, said hydrophobic group has a log P value >0, such as including from 0.05, e.g. 0.1, 0.5, 1.0, 1.5, 2.0, 2.5, 3.0, 3.5, 4.0, 4.5, 5.0, 5.5, 6.0, 6.5. As mentioned above, denatured macromolecules, as well as aggregates of a macromolecule are more hydrophobic than an intact, non-denatured, non-aggregated macromolecule. Aggregates therefore bind to the hydrophobic group of the ligand to a higher extent than a non-aggregated macromolecule.

The hydrophobic group of the ligand may be non-polar, aromatic, and/or aliphatic.

Non-limiting examples of hydrophobic groups which are suitable for use in the method according to the present disclosure comprise the side chain of an amino acid selected from the group consisting of alanine, valine, leucine, isoleucine, methionine, phenylalanine, tryptophan, and tyrosine, or a hydrophobic derivative of said side chain, preferably the side chain of an amino acid selected from the group consisting of phenylalanine, tryptophan, and tyrosine.

The chemical structures of the side chains (i.e. hydrophobic groups) of said amino acids are shown below in Table 1.

TABLE 1
Chemical structure of side chains of amino acids mentioned above

| Amino acid | |
|---|---|
| 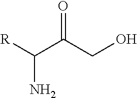 | 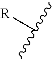 |
| alanine | 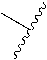 |
| valine | 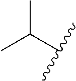 [SMILES: *C(C)C] |
| leucine | 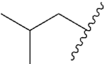 [SMILES: *CC(C)C] |
| isoleucine | 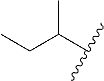 [SMILES: *C(C)CC] |
| methionine | 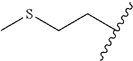 [SMILES: *CCSC] |
| phenylalanine | 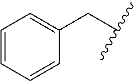 [SMILES: *Cc1ccccc1] |
| tryptophan | 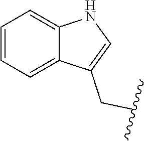 [SMILES: *Cc1c[nH]c2ccccc12] |
| tyrosine | 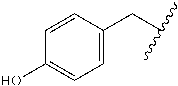 [SMILES: *Cc1ccc(O)cc1] |

The term “derivative” is intended to mean a compound that is derived from a similar compound by a chemical reaction or a compound that at least theoretically can be formed from the precursor compound (Oxford Dictionary of Biochemistry and Molecular Biology, 2003, Oxford University Press, ISBN 0-19-850673-2, https://archive.org/details/isbn_9780198506737). Herein, a “hydrophobic derivative” is to be understood as a derivative that results in a hydrophobic group having a log P value>0.

According to another non-limiting example, the hydrophobic group may be comprised by an amino acid, which has a hydrophobic side chain.

Amino acids are organic compounds that contain amine (—NH2) and carboxyl (—COOH) functional groups, along with a side chain (R group) specific to each amino acid. The chemical properties of an amino acid are largely dictated by the nature of its variable R group.

A “non-polar amino acid” belongs to a class of amino acids in which the variable R group is comprised of mostly hydrocarbons; the amino acids cysteine and methionine also feature a sulphur atom, but (due to its similar negativity to carbon) this does not confer any polar properties to either of these amino acids. The non-polar amino acids include alanine, valine, leucine, isoleucine, and phenylalanine. According to some classifications also glycine, proline, cysteine and methionine belong to the group of non-polar amino acids.

The term “aromatic amino acid” is defined herein as an amino acid which has aromatic side chains. A side chain is aromatic when it contains an aromatic ring system. The strict definition has to do with the number of electrons contained within the ring. Generally, aromatic ring systems are planar, and electrons are shared over the whole ring structure. Tryptophan, tyrosine, and phenylalanine are aromatic amino acids.

An “aliphatic amino acid” is an amino acid containing an aliphatic side chain functional group. Aliphatic amino acids are non-polar and hydrophobic. Hydrophobicity increases as the number of carbon atoms on the hydrocarbon chain increases. Most aliphatic amino acids are found within protein molecules, i.e. in the interior of the protein molecules, if the protein molecules are intact, i.e. non-degraded and non-aggregated. Among the 20 essential amino acids, the true aliphatic amino acids are alanine, valine, leucine, isoleucine. According to some classifications, also proline belongs to the aliphatic amino acids. Strictly speaking, aliphatic implies that the protein side chain contains only carbon or hydrogen atoms. However, it is convenient to consider also methionine in this category. Although its side-chain contains a sulphur atom, it is largely non-reactive, meaning that methionine effectively substitutes well with the true aliphatic amino acids.

Herein, the term “hydrophobic amino acid” is intended to mean an amino acid which has hydrophobic side chains, i.e. side chains that repel water. For this reason, one generally finds these amino acids buried within the hydrophobic core of a protein, or within the lipid portion of a membrane. Non-polar amino acids, aromatic amino acids, and aliphatic amino acids, as defined above, all have hydrophobic side chains, and thus may alternatively be called hydrophobic amino acids. Consequently, for the purpose of the present disclosure, the following amino acids are considered to be hydrophobic amino acids: alanine, valine, leucine, isoleucine, methionine, phenylalanine, tryptophan, and tyrosine.

The chemical structures of said amino acids are shown below in Table 2.

TABLE 2
Chemical structure of amino acids comprising hydrophobic groups

| Amino acid | 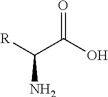 |
|---|---|
| alanine | 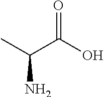 |
| valine | 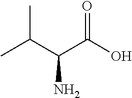 |
| leucine | 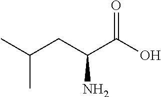 |
| isoleucine | 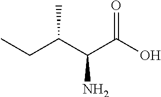 |
| methionine | 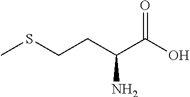 |
| phenylalanine | 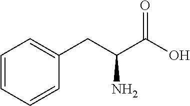 [SMILES: N[C@@H](Cc1ccccc1)C(=O)O] |
| tryptophan | 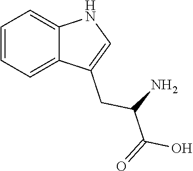 |
| tyrosine | 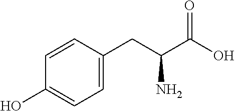 [SMILES: N[C@@H](Cc1ccc(O)cc1)C(=O)O] |

Accordingly, further non-limiting examples of a hydrophobic group which may be used in the method of the present disclosure are hydrophobic groups comprised by a naturally occurring amino acid selected from the group consisting of alanine, valine, leucine, isoleucine, methionine, phenylalanine, tryptophan, tyrosine, or a hydrophobic derivative thereof, preferably a naturally occurring amino acid selected from the group consisting of phenylalanine, tryptophan, and tyrosine.

Alternatively, the hydrophobic group is comprised by a non-naturally occurring amino acid, which has one or more hydrophobic side chains. Non-limiting examples thereof are non-naturally occurring hydrophobic derivatives of alanine, valine, leucine, isoleucine, methionine, phenylalanine, tryptophan, and tyrosine. The term “hydrophobic derivative” is defined elsewhere herein.

As mentioned above, the method of the present disclosure comprises performing at least one of steps (c)(i) and (c)(ii), wherein step (c)(i) comprises comparing the at least one parameter determined in step (b) with the corresponding parameter(s) determined for at least one additional sample, and wherein step (c)(ii) comprises determining at least one parameter related to aggregate(s) of the macromolecule(s).

According to the present disclosure, the at least one additional sample in step (c)(i) may be a second sample, for which the presence, fraction, concentration, and/or amount of macromolecule(s) in the form of aggregate(s) is to be determined. A number of samples, including the first sample and several additional samples, such as 2, 3, 4, 5, 6, 7, 8, 9, 10 or more samples in total, may be analysed and compared to each other, thereby qualitatively determining the presence, fraction, concentration, and/or amount of macromolecule(s) in the form of aggregate(s) in said samples. In other words, the relative fraction, concentration and/or amount of aggregate in a sample may thereby be determined.

The relative concentration of aggregate may be a sufficient measure for example when conducting stability studies of a macromolecule. Accordingly, the present disclosure further provides a method for determining of the stability of a macromolecule, comprising performing the above-described method, wherein the at least one additional sample is a second sample for which the presence, fraction, concentration, and/or amount of macromolecule(s) in the form of aggregate(s) is to be determined, and further comprising subjecting the first sample to a first external condition and subjecting the second sample to a second external condition, wherein the first condition and the second condition differ from each other. For example, the first condition and the second condition may comprise different storage conditions, such as different storage time periods, different storage buffers, different temperatures, and/or different levels of humidity. Thereby, it is possible to determine which storage conditions are better than others, without the need for quantitative determination of the fraction, concentration, and/or amount of aggregates of macromolecule.

However, in other applications of the presently disclosed method, it is of importance to obtain a quantitative measure of the fraction, concentration, and/or amount of aggregates of macromolecule in a sample. Consequently, as an alternative or in addition to the above-described embodiment in which the at least one additional sample in step (c)(i) is a second sample, for which the presence, fraction, concentration, and/or amount of macromolecule(s) in the form of aggregate(s) is to be determined, the at least one additional sample in step (c)(i) may be a control sample having a pre-determined (i.e. known) presence, fraction, concentration, and/or amount of aggregate(s) of the macromolecule(s). A number of control samples, such as 2, 3, 4, 5 or more, may form a standard (also called a standard curve), to which the sample to be analysed is compared. If the fraction, concentration, and/or amount of aggregate(s) of the macromolecule(s) in the control sample(s) or standard has been determined quantitatively, the fraction, concentration, and/or amount of aggregate(s) of the macromolecule(s) in the sample to be analysed may consequently also be determined quantitatively. In other words, the absolute fraction, concentration and/or amount of aggregate in a sample may thereby be determined.

Under certain conditions, the observed rate of binding between a macromolecule and a ligand may be used directly to quantitatively determine the concentration of the macromolecule in the sample, without the use of any standard. This type of assay is referred to as Calibration Free Concentration

Analysis, CFCA. A detailed description of the calculation or determination of parameters required for CFCA analysis, including but not limited to the diffusion coefficient for the macromolecule in the sample, is found in Pol E et al., Evaluation of calibration-free concentration analysis provided by Biacore™ systems, Analytical Biochemistry, 510: 88-97, 2016, which is incorporated herein by reference in its entirety. CFCA may be used to obtain both absolute and relative measurements of macromolecule concentration.

Accordingly, as an alternative to step (c)(i) described above, the method of the present disclosure may comprise performing step (c)(ii), which comprises determining at least one parameter related to aggregate(s) of the macromolecule(s). More particularly, step (c)(ii) of the presently disclosed method may comprise determining a diffusion coefficient for the aggregate(s) and the molecular weight of the aggregate(s), as required for CFCA analysis.

The method of the present disclosure may further comprise determining the at least one parameter (e.g. kinetic parameter) for the interaction of the sample with the sensing surface continuously or intermittently during a time period as a function of elapsed time.

Alternatively, or additionally, the method may further comprise varying the temperature at the sensing surface during a time period, during which at least one parameter (e.g. kinetic parameter) is determined for the interaction of the sample with the sensing surface.

In the presently disclosed method, to be capable of binding (i.e. being immobilised) to the sensing surface, the ligand comprises an amino group, an amine, or a carboxyl group.

Another aspect of the present disclosure involves the use of the above-described method, according to any one of its embodiments, for the determination of the stability of a macromolecule.

Further provided herein is the use of the above-described method, according to any one of its embodiments, for the quantitative or absolute determination of the fraction, concentration, and/or amount of aggregated macromolecule(s) in a sample, as well as the use of the above-described method, according to any one of its embodiments, for the qualitative or relative determination of the fraction, concentration, and/or amount of aggregated macromolecule(s) in a sample.

According to yet another aspect, the present disclosure provides an interaction analysis sensor arranged to perform the steps of at least one of the methods described herein. In particular, said interaction analysis sensor comprises a sensing surface, on which sensing surface is immobilised a ligand comprising a hydrophobic group, which is capable of specific binding interaction with aggregates of macromolecule and which has higher affinity for an aggregated macromolecule than for a non-aggregated macromolecule. The interaction analysis sensor is preferably a sensor for performing surface plasmon resonance.

According to yet another aspect, the present disclosure provides an interaction analysis sensor 1 comprising a sensing surface 2, on which sensing surface 2 is immobilised a ligand 3 comprising a hydrophobic group, which is capable of specific binding interaction with aggregates of macromolecule and which has higher affinity for an aggregated macromolecule than for a non-aggregated macromolecule.

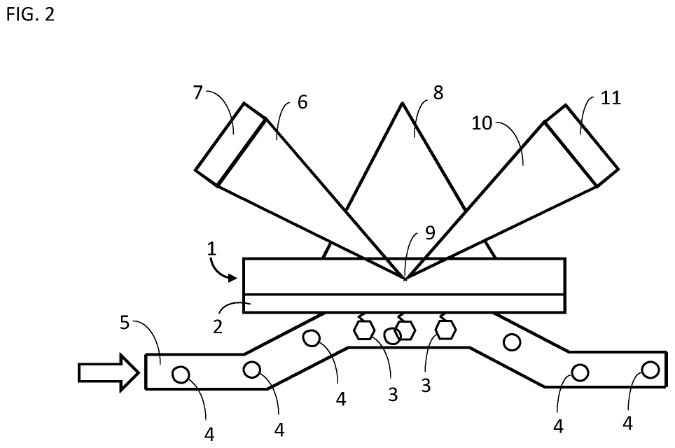

FIG. 2 schematically shows a non-limiting example of an interaction analysis sensor 1, which may be used to perform the method according to the present disclosure. More particularly, FIG. 2 is a schematic illustration of the BIACORE® system. A sensor 1 (alternatively called a sensor chip) made of glass comprises a sensing surface 2, which is covered by a film of metal and on which ligands 3 comprising hydrophobic groups are immobilised. The ligands 3 are exposed to a sample flow with analytes 4 passing through a flow channel 5. Monochromatic p-polarised light 6 from a light source 7 (LED) is coupled by a prism 8 to the glass/metal interface 9 where the light is totally reflected. The intensity of the reflected light beam 10 is detected by an optical detection unit 11 (photodetector array).

The hydrophobic group of the ligand 3, immobilised on the sensing surface 2 of the interaction analysis sensor 1, has a log P value>0.

Said hydrophobic group of the ligand 3, immobilised on the sensing surface 2 of the interaction analysis sensor 1, may comprise the hydrophobic side chain of an amino acid selected from the group consisting of valine, leucine, isoleucine, phenylalanine, methionine, tryptophan, cysteine, glycine, alanine, tyrosine, histidine, threonine, serine, and proline, or a derivative of one of said hydrophobic side chains.

Further, said hydrophobic group may be comprised by an amino acid, which has one or more hydrophobic side chain(s). The amino acid may be a naturally occurring amino acid selected from the group consisting of valine, leucine, isoleucine, phenylalanine, methionine, tryptophan, cysteine, glycine, alanine, tyrosine, histidine, threonine, serine, and proline, or a derivative thereof.

Alternatively, the amino acid may be a non-naturally occurring amino acid as exemplified elsewhere herein.

The ligand 3, immobilised on the sensing surface 2 of the interaction analysis sensor 1, may comprise an amine, an amino group or a carboxyl group, such that the ligand 3 is capable of binding to the sensing surface 2.

The interaction analysis sensor 1 according to the present disclosure may be used to study various types of macromolecules, including but not limited to proteins or polypeptides, such as antibodies.

The interaction analysis sensor 1 according to the present disclosure may be a biosensor, such as a mass-sensing biosensor, preferably a biosensor based on evanescent wave sensing, especially surface plasmon resonance (SPR).

Unless defined otherwise, all technical and scientific terms used herein have the same meaning as commonly understood by a person skilled in the art related to this invention. Also, the singular forms “a”, “an”, and “the” are meant to include plural reference unless it is stated otherwise.

All publications, patent applications, patents, and other references mentioned herein are incorporated by reference in their entirety.

While the present disclosure has been illustrated in the context of SPR spectroscopy, and more particularly the BIACORE® system, it is to be understood that the present disclosure is not limited to said detection method. Rather, any affinity-based detection method where an analyte binds to a ligand immobilised on a sensing surface may be employed, provided that a change at the sensing surface can be measured which is quantitatively indicative of binding of the analyte to the immobilised ligand thereon.

### EXAMPLE 1

### Introduction

In this Example, experiments demonstrating the kinetic behavior of samples containing different fractions of non-aggregated macromolecule and aggregates of macromolecule are described.

### Instrumentation and Materials

A BIACORE® 8K instrument (GE Healthcare Bio-Sciences AB, Uppsala, Sweden) was used. In this instrument, a micro-fluidic system passes samples and running buffer through eight individually detected flow cells (simultaneously or in series). The sensor chip used was a Series S sensor Chip CM5

(GE Healthcare Bio-Sciences AB) which has a gold-coated surface with a covalently carboxymethyl-modified dextran polymer hydrogel. A mixture of the hydrophobic amino acids phenylalanine and tryptophan was immobilised on the sensor surface as specific binding partner (ligand) for aggregates of the analyte. For calculations, the Biacore Insight control software and the Biacore Insight evaluation software (GE Healthcare Bio-Sciences AB, Uppsala, Sweden), dedicated to the BIACORE® instrument, were used.

The analyte studied was a monoclonal IgG antibody.

The output from the BIACORE® instrument is a sensorgram, which is a plot of detector response (measured in “resonance units”, or “response units”, RU) as a function of time. In general, an increase of 1000 RU corresponds to an increase of mass on the sensor surface of approximately 1 ng/mm².

### Preparation of Samples

A sample containing the non-aggregated (monomeric) monoclonal IgG antibody at a concentration of 50μg/m1 was divided into two parts. The first part of the sample was heated to 65 ° C. for 1 h 15 min to create a degraded antibody and/or aggregates of the antibody in the sample. The second part of the sample was unheated; kept at a temperature suitable for keeping the antibody non-degraded and non-aggregated. Aliquots of the first part (heated) of the sample were mixed with aliquots of the second part (unheated) of the sample at different ratios of non-aggregated antibody to aggregated antibody, thereby creating the following series of samples:
a. 100% aggregated;
b. 75% aggregated, 25% non-aggregated;
c. 50% aggregated, 50% non-aggregated;
d. 25% aggregated, 75% non-aggregated;
e. 100% non-aggregated.

Control experiments for the same series of samples a-e were performed with fluorimetry using the fluorescent probe Sypro orange.

### Results and Conclusions

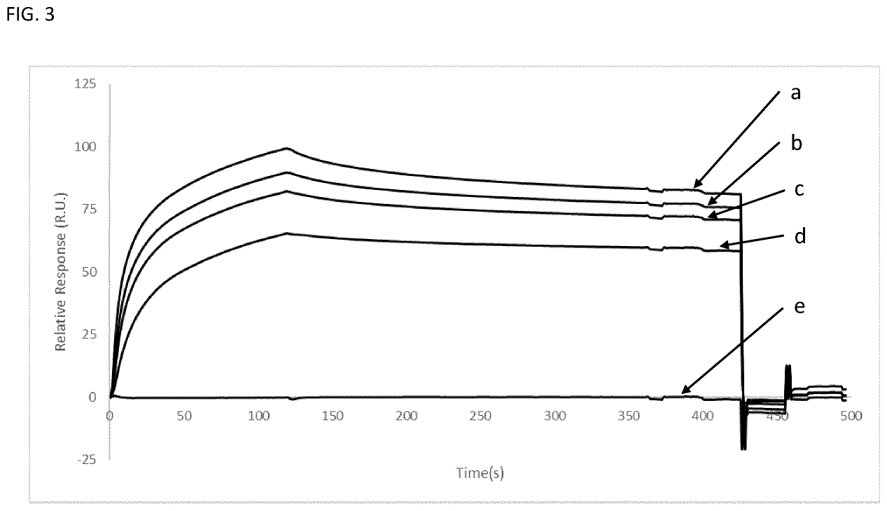

As shown by the association and dissociation curves a-e in FIG. 3, aggregate was bound to the sensing surface of the BIACORE® instrument. Curves a-e of FIG. 3 refer to the series of samples a-e described above under Preparation of samples. The higher amount of aggregated antibody in a sample, the larger the binding response is. The response values of curves a-e have been reference-subtracted.

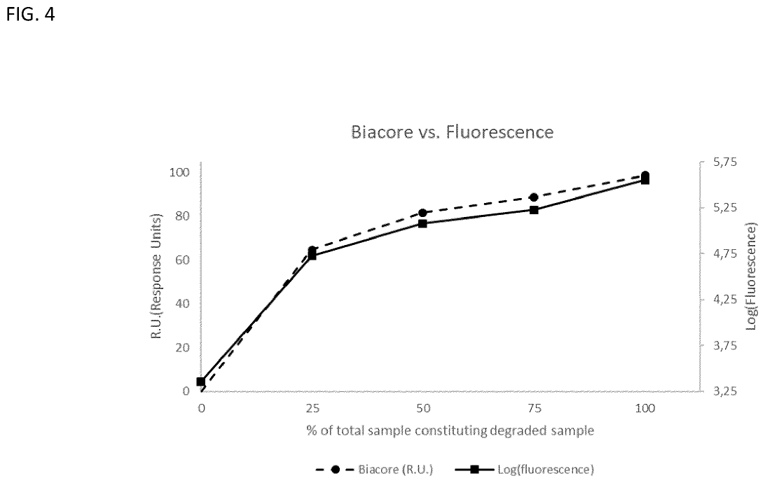

The results of the BIACORE® experiment correlated well with the results of the control experiments performed with fluorimetry, as shown in FIG. 4.

### EXAMPLE 2

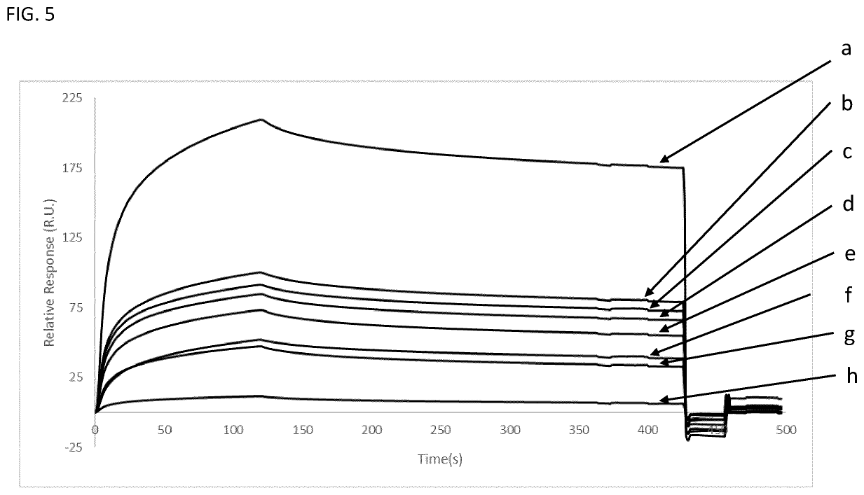

FIG. 5 shows the response curves a-h when injecting an aggregated sample onto a sensing surface comprising various concentrations and ratios of immobilised ligand (i.e. hydrophobic amino acid), as follows:
a. 50 mM tryptophan
b. 50 mM phenylalanine: 25 mM tryptophan
c. 25 mM phenylalanine: 12.5 mM tryptophan
d. 12.5 mM phenylalanine: 6.25 mM tryptophan
e. 6.25 mM phenylalanine: 3.13 mM tryptophan
f. 100 mM phenylalanine
g. 3.13 mM phenylalanine: 1.56 mM tryptophan
h. 1.56 mM phenylalanine: 0.78 mM tryptophan

The different curves are different channels in the Biacore instrument. Six of the curves are immobilised ligand constituting 2:1 molar injection concentration ratios of phenylalanine to tryptophan starting from 1.56:0.78 mM up to 50:25 mM. Two of the curves are immobilised ligand constituting either phenylalanine or tryptophan in inject concentration of either 100 mM (Phe) or 50 mM (Trp) concentrations.

From FIG. 5, it is obvious that with increasing amount of immobilised ligand (i.e. hydrophobic amino acid), there is also an increased response/binding of the aggregates. Herein it is shown that high tryptophan concentrations mediate the highest binding capacity of aggregates while phenylalanine provides lower capacity. Increasing inject concentrations of ligand mixtures provides increased binding capacity.

For comparison, an unheated sample was injected onto the sensing surface comprising various concentrations and ratios of immobilised ligand as defined in a-h above. The only difference between the unheated sample and the “aggregated” sample is that the unheated sample did not go through heat treatment in order to induce aggregates. No binding could be detected except for in the 50 mM tryptophan channel where the approximate binding was 2-2.5 R.U., corresponding to a binding level of approximately 1-1.2% of the previously shown aggregated sample. This does not have to imply that this surface binds non-aggregated sample but might suggest that a small part of the unheated sample constitutes aggregates or hydrophobic surface tendencies as a result of degradation.

### EXAMPLE 3

An experimental design is performed with the same antibody as in Examples 1 and 2, with the following variations:
(a) Testing of different ligands immobilised on the sensing surface of the interaction analysis sensor, i.e. ligands comprising different hydrophobic groups, such as different hydrophobic amino acids or different side chains of hydrophobic amino acids.
(b) Determination of affinity of different ligands towards different multimeric forms of the antibody.

### EXAMPLE 4

An experimental design is performed as in Examples 1 and 2 with other macromolecules than in Examples 1 and 2, for example a protein not being an antibody, e.g. a protein of 50-100 kDa.
(a) Preparing aggregated samples by subjecting samples to heating sessions of various lengths, e.g. 15 min, 30 min, 60 min, 75 min, 200 min. The heating time periods chosen will depend on the stability of the macromolecule.
(b) Analyzing the samples prepared in (a) by use of a surface coated with ligand comprising a hydrophobic group in accordance with the present disclosure. The above procedure may also be complemented by subjecting identical samples to real-time analyses like DSC (Differential scanning calorimetry) and/or Fluorimetry for comparison. This experiment is done to study the effects at various levels of aggregation, from monomeric to fully aggregated.

### EXAMPLE 5

A sample containing the non-aggregated (monomeric) monoclonal IgG antibody at a concentration of 50μg/ml was divided into ten parts. Each part of the sample was heated at 65 ° C. for a set time according to TABLE 3.

TABLE 3

| | Incubation | Sample | Sample | Sample | Sample | Sample | Sample | Sample | Sample | Sample | Sample |
|---|---|---|---|---|---|---|---|---|---|---|---|
| Analyte | Temperature | 1 | 2 | 3 | 4 | 5 | 6 | 7 | 8 | 9 | 10 |
| mAb2 | 65 C. | 0 min | 15 min | 30 min | 60 min | 90 min | 120 min | 150 min | 190 min | 220 min | 250 min |

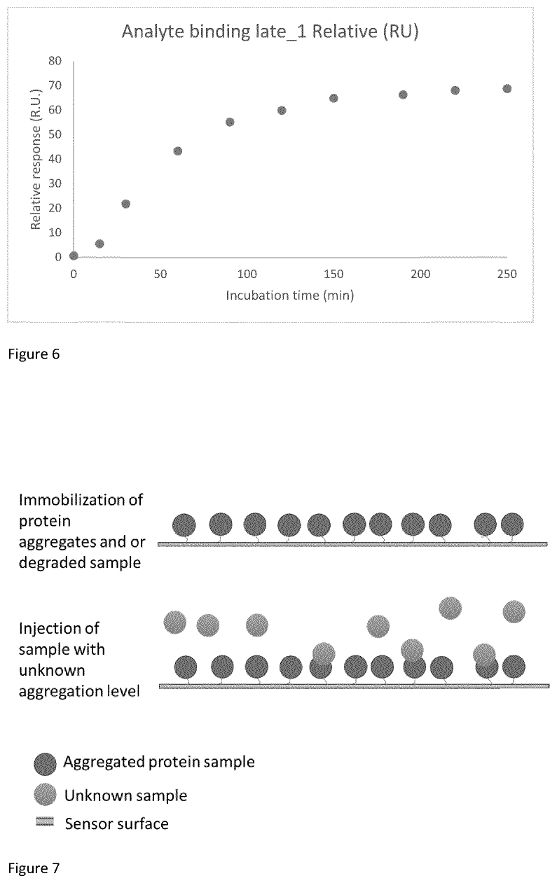

The samples were analyzed with Biacore on a sensor surface immobilized with amino acids. FIG. 6 show the response from each incubation time, where it is seen that the response is increasing with higher incubation time until a plateau is reached. This indicates that the more the sample is degraded the higher affinity it has to the surface.

By using this procedure this it is also possible to measure the level of degradation in a sample by injecting a “true” sample from e.g. cultivation and measure its response in order to find the level of degradation and aggregation.

### EXAMPLE 6

Since aggregation in some aspect is caused by self-affinity it could be possible to immobilize a degraded aggregated sample onto a sensor surface as in FIG. 7. Samples having unknown aggregation levels can be injected over the sensor surface and have the possibility to bind selectively to the degraded proteins on the surface, which would result in an increase in signal response. In this way it is possible to create an aggregation detection method based on the samples at hand and of most interest. Compared to the other method where amino acids are immobilized onto the sensor surface, this method is not as generic and could probably only detect protein aggregates of the same sort. See FIG. 7 for an example how the methodology could be performed.

1. A method for determining of aggregates comprising one or more macromolecules, in a first sample potentially comprising aggregates of the macromolecule(s), comprising the steps of:
a) contacting a first sample with a sensing surface of an interaction analysis sensor, said sensing surface having immobilised thereon a ligand comprising a hydrophobic group, which is capable of increased binding interaction with aggregates of macromolecule(s) compared to non-aggregated macromolecule(s);
b) determining at least one parameter for the interaction of the first sample with the sensing surface;
c) performing at least one of steps (i) and (ii):
i. comparing the at least one parameter determined in step (b) with the corresponding parameter(s) determined for at least one additional sample potentially comprising aggregates of the macromolecule(s);
ii. determining at least one parameter related to aggregate(s) of the macromolecule(s); and
d) determining the presence, fraction, concentration, and/or amount of macromolecule(s) in the form of aggregate(s) in the first sample.

2. The method of claim 1, wherein the hydrophobic group has a log P value>0.

3. The method of claim 1, wherein the hydrophobic group comprises the side chain of an amino acid selected from the group consisting of alanine, valine, leucine, isoleucine, methionine, phenylalanine, tryptophan, and tyrosine, or a hydrophobic derivative of said side chain, preferably the side chain of an amino acid selected from the group consisting of phenylalanine, tryptophan, and tyrosine.

4. The method of claim 1, wherein the hydrophobic group is comprised by an amino acid, which has one or more hydrophobic side chains.

5. The method of claim 4, wherein the amino acid is a naturally occurring amino acid selected from the group consisting of alanine, valine, leucine, isoleucine, methionine, phenylalanine, tryptophan, and tyrosine, or a hydrophobic derivative of said side chain, preferably an amino acid selected from the group consisting of phenylalanine, tryptophan, and tyrosine.

6. The method of claim 4, wherein the amino acid is a non-naturally occurring amino acid.

7. The method of claim 1, wherein the at least one additional sample in step (c)(i) is a control sample having a known presence, fraction, concentration, and/or amount of aggregate(s) of the macromolecule(s).

8. The method of claim 1, wherein the at least one additional sample in step (c)(i) is a second sample, for which the presence, fraction, concentration, and/or amount of macromolecule(s) in the form of aggregate(s) is to be determined.

9. The method of claim 1, wherein step (c)(ii) comprises determining a diffusion coefficient for the aggregate(s) and the molecular weight of the aggregate(s).

10. The method of claim 1, wherein the macromolecule is a protein or a polypeptide, such as an antibody.

11. The method of claim 1, wherein said ligand comprises an amino group or a carboxyl group, which is capable of binding to the sensing surface.

12. The method of claim 1, wherein the interaction analysis sensor is a biosensor.

13. The method of claim 1, wherein the biosensor is a mass-sensing biosensor, preferably a biosensor based on evanescent wave sensing, especially surface plasmon resonance (SPR).

14. The method of claim 1, comprising determining the at least one parameter for the interaction of the sample with the sensing surface continuously or intermittently during a time period as a function of elapsed time.

15. The method of claim 1, wherein the at least one parameter is a kinetic parameter, optionally wherein said kinetic parameter is selected from the group consisting of the association rate, the dissociation rate, the association rate constant (kₐ), the dissociation rate constant (k_{d}), the affinity constant (K_{A}), and the dissociation constant (K_{D}).

16. The method of claim 1, further comprising varying the temperature at the sensing surface during said time period.

17. A method for determining of the stability of a macromolecule, comprising performing the method of claim 8, and further comprising subjecting the first sample to a first external condition and subjecting the second sample to a second external condition, wherein the first condition and the second condition differ from each other.

18. The method of claim 17, wherein the first condition and the second condition comprise different storage conditions, such as different storage time periods, different storage buffers, different temperatures, and/or different humidity.

19. Use of the method of for the determination of the stability of a macromolecule.

20. Use of the method of claim 1, for the determination of degradation of protein in a sample.

21. Use of the method of claim 1, for the determination of biological activity of a protein drug or a polypeptide drug.

22. Use of the method of claim 1, for the quantitative determination of the fraction, concentration, and/or amount of aggregated macromolecule(s) in a sample.

23. Use of the method of claim 1, for the qualitative determination of the presence, fraction, concentration, and/or amount of aggregated macromolecule(s) in a sample.

24. An interaction analysis sensor for use in a method according to claim 1, said interaction analysis sensor comprising a sensing surface, on which sensing surface is immobilised a ligand comprising a hydrophobic group, which is capable of increased binding interaction with aggregates of macromolecule(s) compared to non-aggregated macromolecule(s).

25. An interaction analysis sensor comprising a sensing surface, on which sensing surface is immobilised a ligand comprising a hydrophobic group, which is capable of increased binding interaction with aggregates of macromolecule(s) compared to non-aggregated macromolecule(s).

26. The interaction analysis sensor of claim 24, wherein said hydrophobic group has a log P value>0.

27. The interaction analysis sensor of claim 24, wherein said hydrophobic group comprises the side chain of an amino acid selected from the group consisting of alanine, valine, leucine, isoleucine, methionine, phenylalanine, tryptophan, and tyrosine, or a hydrophobic derivative of said side chain, preferably the side chain of an amino acid selected from the group consisting of phenylalanine, tryptophan, and tyrosine.

28. The interaction analysis sensor claim 24, wherein said hydrophobic group is comprised by an amino acid, which has one or more hydrophobic side chain(s).

29. The interaction analysis sensor of claim 28, wherein the amino acid is a naturally occurring amino acid selected from the group consisting of alanine, valine, leucine, isoleucine, methionine, phenylalanine, tryptophan, and tyrosine, or a hydrophobic derivative thereof, preferably an amino acid selected from the group consisting of phenylalanine, tryptophan, and tyrosine.

30. The interaction analysis sensor of claim 28, wherein the amino acid is a non-naturally occurring amino acid.

31. The interaction analysis sensor of claim 24, wherein the macromolecule is a protein or a polypeptide, such as an antibody.

32. The interaction analysis sensor of claim 24, wherein said ligand comprises an amino group or a carboxyl group, which is capable of binding to the sensing surface.

33. The interaction analysis sensor of claim 24, wherein the interaction analysis sensor is a biosensor.

34. The interaction analysis sensor of claim 33, wherein the biosensor is a mass-sensing biosensor, preferably a biosensor based on evanescent wave sensing, especially surface plasmon resonance (SPR).
